(19) United States
(12) Patent Application Publication
(10) Pub. No.: US 2018/0207422 A1
(43) Pub. Date: Jul. 26, 2018

(54) Instrument for regulating vital energy zen qi

(71) Applicant: Zhen Li, Toronto (CA)

(72) Inventor: Zhen Li, Toronto (CA)

(21) Appl. No.: 15/703,958

(22) Filed: Sep. 13, 2017

(30) Foreign Application Priority Data
Oct. 14, 2016 (CN) 201610907267.8

(51) Int. Cl. A61N 1/36; A61H 23/02; A61N 1/02; A61H 39/02

(52) U.S. Cl. CPC A61N 1/3615; A61H 39/02; A61N 1/025; A61H 23/02

(57) ABSTRACT

The invention discloses an device for regulating vital energy—Zen Qi ( [symbol] ), which relates to the field of biotechnology, which comprises two electrodes and an electronic circuit generating output, where the positive and negative electrode of the output are placed respectively close to or contact with the yin and yang sides of the organism; This device include: the power supply section, the current control section, the data storage and display section, the constant current section, the over-current protection section, the over-voltage protection section and the current detection section. By using real-time current detection and over-current and over-voltage protection, this device ensures the safety of organisms. The functions of the electronic circuit as described above can be achieved by discrete components or by integrated circuit. If it uses integrated circuit, it can greatly save a lot of discrete components, reducing the size of the device and improve its reliability. After using this device many people have increased saliva, improved digestion, memory, and immunity system, improved health condition, etc., the result are the same as: vital energy has been greatly replenished. This device is worth to be promoted to the world.

### TECHNICAL FIELD

This invention belongs to the field of biotechnology, it specially discloses an device for regulating Vital energy Zen Qi ( [symbol] ).

### BACKGROUND TECHNIQUE

Vital energy Zen Qi^{[2]} is the vital energy, come from parent when organism born and it can be replenished after born, Taoism^{[1]} believes that Vital energy Zen Qi can be achieved by practice of “Physical and Mental Comprehensive Cultivation”^{[3]}, or by qigong. According to the theory of traditional Chinese medicine, vital energy Zen Qi is the most basic material to maintain organism life; all human life depends on the vital energy Zen Qi. (From Baidu Encyclopedia http://baike.baidu.com/). “The Yellow Emperor's Classic of Internal Medicine” and the ancient and modern Chinese medicine have a lot of descriptions about vital energy Zen Qi. For example: “The Yellow Emperor's Classic of Internal Medicine. Simple questions. The Universal Truth”: “Previously people led a calm and honest existence, detached from undue desire and ambition, thus vital energy Zen Qi flow freely; attention is always kept inside, where illness can come from?”. Traditional Chinese medicine And “The Yellow Emperor's Classic of Internal Medicine” also believes that the biological head belongs to the yang, the foot belongs to the yin, back belongs to yang, abdomen belongs to yin.

“The Yellow Emperor's Classic of Internal Medicine •Spiritual Pivot” pointed out: Vital energy Zen Qi, comes from the universe and combines with nutrition then run within organism.

“The Yellow Emperor's Classic of Internal Medicine ⋅Simple questions ⋅Pathogens” says: Vital energy Zen Qi is the vital material running in meridians. It points out that vital energy Zen Qi is energy running in meridians. Thus meridians are paths of vital energy Zen Qi, vital energy Zen Qi is the activities of meridians. Vital energy Zen Qi is so important for human and other organism. If Vital energy Zen Qi is strong then organism will be health, otherwise, Vital energy Zen Qi is weak then organism will be sick.

Actually Vital energy Zen Qi is movement of electrons in organism; the meridian is the path of movement of electrons in organism. Vital energy Zen Qi comes from to food and breathing of lung, as well as conduction of skin.

The existing method of obtaining vital energy Zen Qi: through Qigong, meditation, cultivation of Buddhism and Taoism and so on exercise methods, now there is no device to help organism obtain vital energy Zen Qi. This invention is to solve this problem by providing such a device for obtaining vital energy Zen Qi.

### INVENTION DETAIL

To achieve the above goal, the present invention provides the following technical solutions: The device comprises: positive and negative electrodes for output, a power supply section, a current control section, a data storage and display section, a constant current section, an over-current protection section, An over-voltage protection section, and a current detection section, which power supply section rectifies an alternating current or a battery or a photocell to DC to supply power to other sections of the device. By real-time current over-current protection and over-voltage protection section, this device gives double protection to ensure safety of organism. This device gives organism a non pulse DC current that is less than 5 mA.

Preferably, the positive and negative output electrodes are respectively approaching or contacting the yang and yin parts of the organism, so that the device and the organism form a complete electrical circuit. The two output electrodes may be of different shapes, such as a pad in a plane, such as a needle if not in a plane.

Preferably, the positive electrode is adjacent to or in contact with the head of a human or organism, and the negative electrode approaches or contacts the foot of a human or organism so that the human body or organism and the device form a complete electrical circuit. Head is defined as positive part, foot as negative parts on the Traditional Chinese medicine.

Preferably, the power supply section rectifies an alternating current or battery or photocell to direct current for powering other parts of the device.

Preferably, the constant current section produces a constant current of less than 5 milliamps for output.

Preferably, current control section is realized by a computer to control current, which receiving a command from a user, setting target current, detecting the current, and providing parameters for the constant current section.

Preferably, the data storage and display section is for storing real-time time or data for various treatment plans of different users.

Preferably, the over-current protection section limits the current flowing through each of the organisms to no more than 5 mA.

Preferably, the over-voltage protection section is used to limit the voltage to no more than 499.99 volts (for the human body), for animals, depending on the height of the animal.

Preferably, the current detection section detects the current flowing through the living body in real time.

The beneficial of this technical proposal is:

The functions of the circuit as described above can be achieved by discrete components, or by a computer plus discrete components. If you use a computer plus discrete components, can greatly save a lot of discrete components, reduce the size of the device and improve its reliability.

### BRIEF DESCRIPTION OF THE DRAWINGS

Following figures will show this technique proposal in further detail:

FIG. 1 is a block diagram of the device of this invention; it describes the connection between the various sections and how this device connects to the organism.

FIG. 2 is a schematic of circuit of this device. It describes an example of implementation. The square wave PWM of the microcomputer switches on and off of transistor Q4, thus control charge and discharge of inductance L2 and capacitance C7. Another output of the microcomputer controls on and off of Q1, thus controls the output of the device to the organism.

FIG. 3 is a microcomputer software programming flow chart.

### EXAMPLE OF IMPLEMENTATION

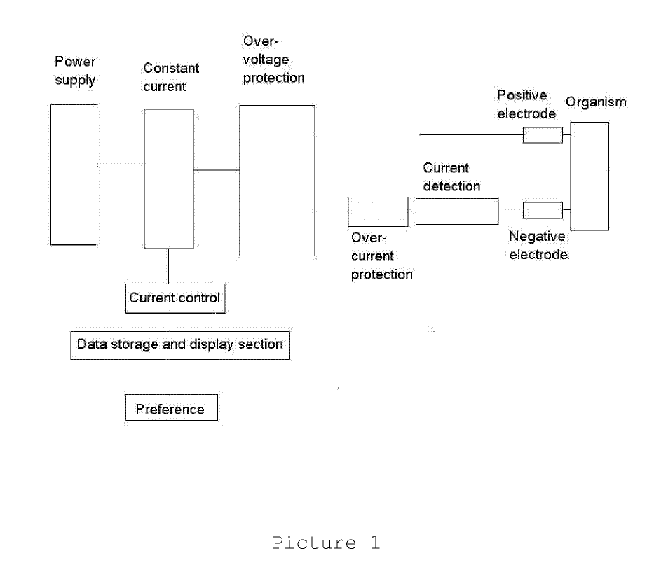

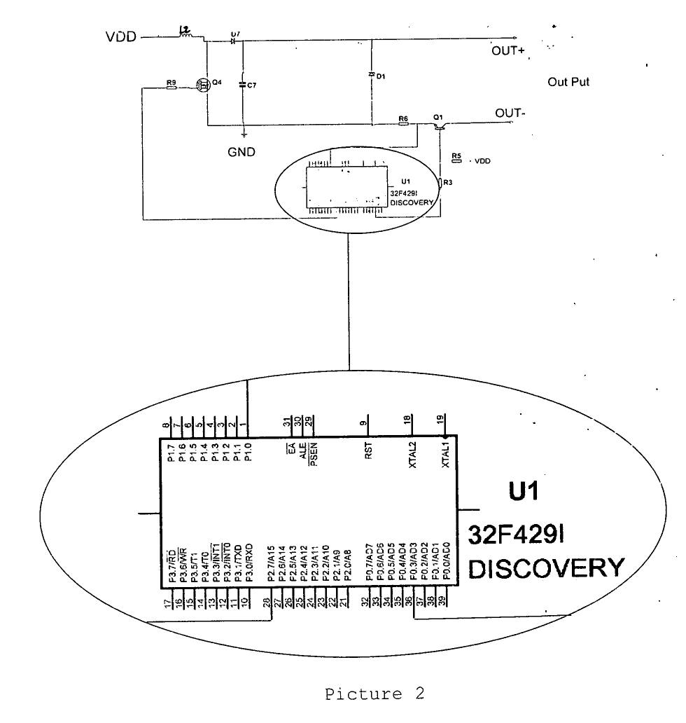

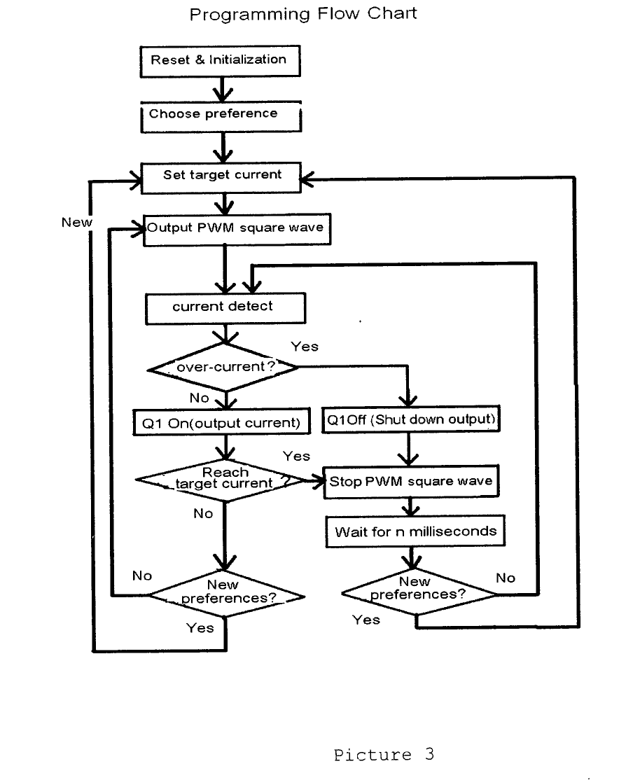

An example of implementation of this device will be descript as follow, and it is shown in figures: FIG. 1, FIG. 2 and FIG. 3 show an implementation of this device according to the present invention. The safe current through which the human body can flow is 10 mA, so this invention takes only half of the safe current; the current is limited to 5 mA or less.

As shown in FIG. 1, the device comprises a positive and negative electrode, a power source section, a current control section, data storage and display section, a constant current section, an over-current protection section, an over-voltage protection section, a current detection section, the device supplies a DC current to organism which is less than 5 mA.

Microcomputer 32F429IDISCOVERY is a very comprehensive and powerful computer, with the latest touch-screen technology, and sufficient memory, and multiple input and output ports. The built-in touch screen can display and key in user's instructions. It can make this device very simple by saving a lot of discrete components.

Referring to FIGS. 1 and 2, the power supply section rectifies alternating current or battery or photocell to direct current for powering other parts of the device. The constant current section produces a constant current of less than 5 mA for output. Current control section is using the 32F429IDISCOVERY microcomputer to control the target current by accepting the user's instructions, setting target current, providing waveform to constant current section, and providing signal of on or off to the over-current protection section and the over-voltage protection section. The Data storage and display section also uses the 32F429IDISCOVERY microcomputer to store and display real time and a variety of treatment plan for different user. The positive electrode of the output approaches or contacts the head of the human body, and the negative electrode approaches or contacts the foot of the human body so that the device and the organism form a complete electrical circuit.

As shown in FIG. 2 and FIG. 3, 32F429IDISCOVERY microcomputer generates a controllable waveform, which controls the on and off of transistor Q4, thus controls the charge of capacitor C7 by inductor L2, the length of the waveform controls the charging time of capacitor, generates a DC potential in the C7, providing a constant value of the output current for the organism. Microcomputer 32F429IDISCOVERY detects real-time current through organism by the resistor R6, when the current exceeds a specific target value, the microcomputer 32F429IDISCOVERY control the gate voltage of transistor Q4, the gate voltage then is set to low, thus the transistor Q4 is turn off, and the inductor L2 stop to charge the capacitor C7. On the hand, when the current is less than a specific target value, the microcomputer 32F429IDISCOVERY generate a specific length of the square wave PWM to the gate of transistor Q4, making the capacitor C7 charged by inductor L2. Thus it ensures that the current flowing through the organism is near the specific target value.

The over-current protection section is used to limit the current flowing through each organism to no more than 5 mA. The principle is as follows: It consists of resistors R6, R3, R5 and transistor Qi and 32F429IDISCOVERY microcontroller. 32F429IDISCOVERY detect real-time current flowing through the organism by resistor R6, when the current reaches a certain value, the microcontroller 32F429IDISCOVERY control the voltage of transistor base, the voltage is set to low, thus the transistor Q1 is turn off. This ensures the current no more than a certain value.

The over-voltage protection section is used to limit the voltage to no more than 499.99 volts. It consists of a 499.99 zener diode D1 which is connected parallel on the output to ensure that the voltage does not exceed this voltage.

Current detection session consists of resistor R6, it provides Microcomputer 32F429IDISCOVERY a voltage that is proportional to current, 32F429IDISCOVERY read voltage value then converts into the current value. In the present implementation, R6 is a resistance of 1% accuracy; this ensures the accuracy of detection.

After use this device, many people report that their saliva increase, and their digestive or memory or immunity system have been improved, health condition improved and so on, this is the same as the someone's vital energy have been enhanced, the same as someone's years of Qigong exercise; many practitioners of standing pile, qigong, Buddhist and Taoist feel that their skills had been improved greatly by helping of this device as an supplementary tool. Therefore, this device is worth to be promoted greatly. So that all the people of the world benefit from this device, so that the biological used by human beings also benefit from this device.

It is to be noted that above is only an example of implementation. According to the principle of this invention, any engineer in this field can have many alternative implementations or improvements. But these alternative implementations or improvements are protected by this invention. Such as using other Microcomputer, other ways of over-voltage protection, other ways of over-current protection, other ways of current detection and so on.
[1] Taoism: en.wikipedia.org/wiki/Religion_in_China#Taoism
[2] Vital energy Zen Qi: http://baike.baidu.com/item/%E7%9C%9F%E6%B0%94/6004118?fr=aladdin
[3] Physical and Mental Comprehensive Cultivation: http://baike.baidu.com/view/190157.htm

1. An device for regulating the Vital energy Zen Qi, the device comprising: positive and negative electrodes for output, a power supply section, a constant current section, a current control section, a data storage and display section, an over-current protection section, An over-voltage protection section, and a current detection section, which power supply section rectifies an alternating current or a battery or a photocell to DC to supply power to other sections of the device.

2. A device according to claim 1, wherein positive electrodes is placed close to or contact with the Yang-positive portion of the organisms, and negative electrodes is placed close to or contact with the Yin-negative portion of the organisms.

3. A device according to claim 1, wherein in that said constant current section produces a constant current output of more than 0 but less than 5 mA.

4. A device according to claim 1, wherein said current control section is realized by a computer which is used for controlling the current, receiving a command from a user, setting target current, detecting the current, and providing parameters for the constant current section.

5. A device according to claim 4, wherein the computer is a microcomputer 32F429IDISCOVERY.

6. A device of claim 1, wherein the data storage and display section is used to store and display real time and various treatment plans of different users.

7. A device according to claim 6, wherein the data storage and display section is realized by a computer which is used for storing and displaying real-time.

8. A device according to claim 7, wherein the computer is a microcomputer 32F429IDISCOVERY.

9. A device according to claim 1, wherein the over-current protection section is configured to limit the current flowing through each of the organisms to no more than 5 mA.

10. A device according to claim 1, wherein the current detection section detects a real time current going through the organisms.

11. A device according to claim 1, wherein in the over-voltage protection section is configured to limit the voltage not to exceed 499.99 volts to ensure the safety of the organism.

12. A device according to claim 5, wherein said microcomputer 32F429IDISCOVERY generates a controllable waveform to control the opening and closing of the transistor, which controls the charging time of the capacitor by the length of the waveform, so that it can provide a constant value of the current output.

13. A device according to claim 9, wherein the over-current protection section is composed of resistors R6, R3, R5, a transistor Qi and a microcomputer 32F429IDISCOVERY.

14. A device according to claim 11, wherein said over-voltage protection section is consisted of a Zener diode D1 connected parallel to 2 ends of the output.

15. A device according to claim 10, wherein said current detection section is composed of a resistor R6 which supplies microcomputer 32F429IDISCOVERY a voltage proportional to current
